(19) United States
(12) Patent Application Publication
(10) Pub. No.: US 2007/0264290 A1
(43) Pub. Date: Nov. 15, 2007

(54) Pharmaceutical and/or Cosmetic Care Product, Such as a Mask

(76) Inventor: Anna Chagnaud, Poissy (FR)

Correspondence Address: YOUNG & THOMPSON, 745 SOUTH 23RD STREET, 2ND FLOOR, ARLINGTON, VA, 22202, US

(21) Appl. No.: 10/557,446

(22) PCT Filed: May 19, 2004
(86) PCT No.: PCT/FR04/01243

§ 371 Date: Mar. 5, 2007

(30) Foreign Application Priority Data
May 20, 2003 (FR) 03/06069
Jul. 7, 2003 (FR) 03/08297

(51) Int. Cl. A61K 9/70; A61K 33/00; A61K 38/00; A61P 17/00; A61Q 19/00; A61K 8/02; A61K 8/97

(52) U.S. Cl. 424/401; 424/402; 514/2

(57) ABSTRACT

A pharmaceutical and/or cosmetic care product, such as a mask, intended to be applied to an area of the human body to be treated. The product includes an absorbent support and a preparation for impregnating the support, the preparation containing at least one active principle for the pharmaceutical and/or cosmetic care of the area to be treated. The invention is intended for pharmaceutical and/or cosmetic care applications.

The present invention relates to a pharmaceutical and/or cosmetic care product such as a mask adapted to be applied to the skin, the hair, etc., as well as a process for producing said product.

Pharmaceutical or cosmetic preparations usable as a mask are generally present in the form of a cream or paste to be applied in a layer to the skin, the hair.

The application of these products in a layer generally gives rise to uncertainty as to the effective dosage, the users having the tendency to produce layers that are too thin for purposes of economy or too thick for fear of ineffectiveness.

Thus, persons who use pharmaceutical or cosmetic products in paste or cream form to produce masks must apply them to the region to be treated without controlling the dose of product in a certain manner and hence effectively.

To produce care masks, it is also the current practice to use natural products such as vegetable products such as cereals such as oats, bran, wheat, vegetables such as cucumber, carrots, vegetable oils, fruits such as grapes, apples, mineral products such as clay.

Thus, one can oneself produce a care mask by cutting, grinding, chopping and mixing vegetables and fruits. It is often necessary also to add all the ingredients necessary for the production of a paste applicable in the manner of a mask or poultice. However, this requires a relatively great preparation time as well as time devoted to cleaning and arranging all the utensils used, which greatly reduced the number of people who will produce their own products.

There have also been proposed care masks including active principles contained in natural products. However, even if the product remains as natural as possible, the packaging of the latter requires most often the use of excipients and preservatives to permit these preparations to remain stable for sale.

As a result, those who seek care products for the skin and/or hair such as masks, based on completely natural products without excipients and other preservatives, have no choice other than to prepare themselves their products each time they wish to use them.

There are known from GB 2 281 730, DE 100 00 313, EP 1 228 753 and EP 1 319 396, care products constituted by a sachet including for example herbs for care such as green tea and whose steeping permits release of cosmetic values of said herbs in the manner of infusions.

In FR 2 808 195, there is disclosed a cosmetic patch comprising a lyophilized matrix containing at least one agent of hydrophilic texture and active principles, the whole being lyophilized. The matrix moreover comprises a screen integrated in the matrix or embedded within said matrix. The presence of this hydrophilic texture agent necessary to give strength to the matrix, renders the preparation including active principles the characteristic of being “home made”, even if these hydrophilic texture agents can be selected from natural products.

So as to overcome these drawbacks, the present invention has for its principal object to provide pharmaceutical and/or cosmetic care products such as masks including active principles present in a ready to use form and directly applicable to a region to be treated and in which the active principles are measured.

A secondary object of the invention is to be able to provide care products which have no usual chemical additive and whose packaging permits limiting or omitting the usual number of additives for preservation.

To this end, the invention has for its object a pharmaceutical and/or cosmetic care product such as a mask adapted to be applied to a region of the human body to be treated, characterized in that it is constituted by an absorbent support and a preparation for impregnating said support, including at least one pharmaceutical and/or cosmetic active care principle for the region to be treated.

According to a first embodiment, the preparation including at least one active principle is present in the form of a fluid, pasty or the like preparation, suitable to be applied, preferably in a measured manner, on the support to be impregnated.

Preferably, the product according to the invention is present in the form of a mask directly applicable to the region to be treated thanks to the support and in which the preparation including the active principle is already measured during impregnation of the support.

There is obtained with this embodiment of the invention, a care product that is simple to use and whose effectiveness is optimized, the mask including an effective dosage of the preparation including the active principles, without wasting products.

Preferably, the mask thus constituted is moreover frozen. Preferably, the care product according to the invention is packaged and frozen such that the usual preservative and stabilizing additives are limited, or even omitted, because it is preserved by freezing.

Preferably, a preparation including at least one active principle is produced from natural products such as fresh vegetables, fresh proteinaceous products and/or mineral products, alone or in a mixture. There can also be used products in dehydrated, dried, reduced or powdered form, for example, to produce the impregnation preparation.

Among the vegetables, there can be used fruits, vegetables, algae, plants, flowers, cereals, spices, simples, oils, alcoholic or non-alcoholic beverages, etc. The proteinaceous products that can be used, preferably derive from meat or fish whilst the minerals used are for example clays.

Thus, the vegetable, proteinaceous or mineral products used can be prepared in the form of juice, paste and other preparations suitable to be able to be applied to the support and to impregnate it. Such a preparation being adapted to impregnate the absorbent support, there is not needed any additional texturing agent, which limits the steps of production as well as the cost but also contributes to the quality of the product.

The preparation including at least one active principle can also be constituted by a conventional pharmaceutical and/or cosmetic preparation effective for application to the skin, the hair, which is to say with transcutaneous action.

After the preparation, for example based on fresh vegetables, has impregnated the absorbent support, the mask thus formed is preserved by freezing and sold in frozen form. The product according to this embodiment of the invention can also be packaged, frozen or not, in sealed packaging, preferably under vacuum, or in packaging with a complex structure including protection against light and atmospheric agents.

The shapes and sizes of the mask are defined as a function of the care region to which it is destined. Thus, for example, a facial mask according to the invention will have a substantially oval and incurved shape so as to be applied to the face. The freezing phase preferably permits the mask to preserve this incurved shape, permitting direct application of the mask to the face of the user.

Thus, during use, the mask according to the invention can be used directly frozen or else it can be reheated to different temperatures. It has been noted that surprisingly, the defrosting of the mask according to the invention does not cause deterioration of the mask, the active products remaining perfectly on the support.

Thus, as a function of the type of products used to produce the mask, the effectiveness of the mask can be optimized by proposing preferred effective temperatures. In this way, the application of the mask according to the invention can give sensations of cold, of freshness or else heat, at the choice of the user.

Still further, during defrosting, the active principles contained in the impregnation preparation of the absorbent support are released toward the region to be treated such as the skin or the hair where they act.

According to a second embodiment of the invention, the impregnation preparation including at least one active principle for pharmaceutical and/or cosmetic care is in hydrophilized or dehydrated form.

Preferably, the dehydrated or hydrophilized preparation is applied to the support, preferably in a measured manner, and can be fixed, for example, by spaced stitching, gluing, bending on the surface of said support.

Thus preferably, it suffices to rehydrate the product according to this second embodiment of the invention such that the latter will be ready to use in a manner capable of being used as a mask directly applicable to the region to be treated. Thus, during rehydration, the preparation impregnates the absorbent support which thus retains this preparation and permits good application of the mask to the region to be treated.

There is thus also obtained with this second embodiment of the invention, a care product simple to use and whose effectiveness is optimized, including an effective dose of the preparation comprising the active principles, without wasting products.

The lyophilized or dehydrated form of the preparation including the active principles permits a simple conservation of the product thus constituted, by sealed packaging, if desired under vacuum.

Preferably, a preparation including at least one active principle is made on the basis of natural products such as fresh vegetables, fresh proteinaceous products and/or mineral products, alone or in a mixture, the preparation then undergoing a dehydration or lyophilization treatment.

The preparation can also be made from products already dehydrated or lyophilized.

Among the vegetables, will be used fruits, vegetables, algae, plants, flowers, cereals, spices, simples, oils, alcoholic or non-alcoholic beverages, etc. The proteinaceous products which can be used, derive preferably from meat or fish whilst the minerals used are for example clays. Such a preparation being adapted then to impregnate the absorbent support during rehydration, no additional texturing agent is required, which limits the steps of production as well as the cost but contributes also to the quality of the product.

Preferably, the support and the lyophilized or dehydrated impregnation preparation are enclosed in an envelope.

This envelope is preferably constituted by a breathable material, on at least one surface. Thus, it can be provided that the material be a cloth, such as passes gases, on the two surfaces of the envelope or else the envelope has but one surface of a breathable material, the other surface being a sealing material.

As a modification, the envelope can also be constituted by an absorbent material and during rehydration, the rehydrated preparation contained therein can impregnate said envelope. Similarly, the absorbent support can constitute itself one of the surfaces of the envelope.

The dimensions and shape of the mask are defined as a function of the care region for which it is destined, the envelope being cut to the corresponding shape. Thus, for example, a facial mask according to the invention has a substantially oval shape. The use of an envelope as described can also be applied to the first embodiment of the invention.

During use, the mask according to the invention is used by rehydrating it. This rehydration can be carried out with soft water, seawater, mineral water, milk, soy milk, fruit or vegetable juice, beer, oil/water emulsions, selected as a function of the care properties which can thus be given supplementally to the active principles already contained in the mask.

Thus, as a function of the type of products used to make the mask, the effectiveness of the mask can be optimized by selecting a particular rehydration medium and, for example, by heating or cooling the product. In this way, the application of the mask according to the invention could give sensations of cold, freshness or else heat, at the choice of the user.

The product according to the invention can also be packaged in complexly structured packaging ensuring protection against light and atmospheric agents.

There is accordingly obtained with the invention a mask for pharmaceutical and/or cosmetic care which is easily storable by the user, simple to use, because it suffices to “wet” it to be able to apply it to the region to be treated and which, once used, can be easily removed, the constituents of the mask remaining in a single piece.

In the two preferred embodiments described above, the absorbent support is preferably a fibrous material, for example of cotton, felt, a strongly absorbent cloth.

The absorbent support can also be constituted by a porous material such as natural or synthetic sponge. The support can preferably be constituted by a layer of sponge having an inherent strength to produce a mask.

The absorbent support can also be constituted by a layer of an absorbent material, covered moreover with a breathable material such as gauze, the impregnation of the absorbent material by the preparation taking place through said breathable material. The preparation thus remains in part on the surface of the gauze and if desired another gauze can be secured thereover.

According to a preferred modification, the absorbent support is applied to a base constituting a breathable cloth such as a gauze which lets the preparation pass through, of a film preferably sealed such as plastic, latex, which does not let the preparation pass, on which is glued the absorbent support such as cotton, sponge, for example with the help of a natural cement such as moist flour.

There is thus obtained a support of better strength which needs no synthetic constituent or additive as a texturizing agent.

A care product according to the invention can thus be considered as a cosmetic product whose constituents (active principle or principles) define the cosmetic properties such as hydration, an anti-aging action, an exfoliating action, an astringent action, etc., and/or as a pharmaceutical product whose constituents (active principle or principles) define the curative properties such as soothing cutaneous irritations, sunburn, burns, the treatment of cutaneous afflictions such as acne, eczema, etc.

When the care product according to the invention comprises pharmaceutical active principles, these latter can be adapted for the care of internal organs, by diffusing through the skin during application, for example by application to the chest to care for the bronchia. In the case of the second embodiment of the invention, the dehydrated or lyophilized preparation can thus be present in pulverized form.

Moreover, the mask according to the invention can be perfectly natural, based on fresh products having a stability without any addition of synthetic additives, these products being moreover presented for use on a support preferably likewise of natural material.

There is thus obtained with the invention a mask for pharmaceutical and/or cosmetic care that is easy to store by the user, that is simple to use because it suffices to apply it to the region to be treated and which, once used, can be easily withdrawn, the constituents of the mask remaining in a single piece.

The invention also relates to a process of production of a product according to one or the other embodiment of the invention, in which the absorbent support is impregnated with a preparation including at least one active principle and it is packaged in sealed packaging. In the first embodiment, the product can be frozen before packaging.

1-21. (canceled)

22. Pharmaceutical and/or cosmetic care product such as a mask adapted to be applied to a region of the human body to be treated, constituted by an absorbent support and a preparation for impregnation of said support, including at least one pharmaceutical and/or cosmetic care active principle for the region to be treated, said product impregnation preparation being in lyophilized or dehydrated form, characterized in that the dehydrated or lyophilized preparation is applied to the support and is fixable by spaced stitching, gluing, spaced bending on the surface of said support and in that the support and the impregnation preparation are enclosed in an envelope.

23. Product according to claim 22, characterized in that the absorbent support is a fibrous absorbent material, such as strongly absorbent cotton, felt, cloth.

24. Product according to claim 22, in that the absorbent support is constituted by a porous material such as a natural or synthetic sponge.

25. Product according to claim 22, characterized in that the absorbent support is constituted by a layer of an absorbent material covered moreover with a breathable material such as a gauze, the impregnation of the absorbent material by the preparation taking place through said breathable material.

26. Product according to claim 22, characterized in that the absorbent support is fixed on a base such as a breathable cloth such as a gauze, a sealed film such as plastic, latex, on which is glued the absorbent support.

27. Product according to claim 26, characterized in that the absorbent support is fixed on the base with the help of a natural glue such as wet flour.

28. Product according to claim 22, characterized in that the impregnation preparation including at least one active principle is based on natural products such as fresh vegetables, fresh protein products and/or mineral products.

29. Product according to claim 22, characterized in that the preparation including at least one active principle is based on natural products such as fresh vegetables, oils, fresh protein products and/or mineral products, then having undergone a dehydration or lyophilization treatment.

30. Product according to claim 22, characterized in that the preparation is made from previously dehydrated or lyophilized products, for example in powdered form.

31. Product according to claim 22, characterized in that the envelope is preferably constituted by a breathable material on at least one surface.

32. Product according to claim 31, characterized in that the envelope is preferably constituted by an absorbent material.

33. Product according to claim 32, characterized in that one of the surfaces of the envelope is constituted by the absorbent support itself.

34. Product according to claim 22, characterized in that it is packaged in a sealed package, preferably under vacuum.

35. Product according to claim 32, characterized in that it is packaged in a package with a structure ensuring protection against light and atmospheric agents.

36. Product according to claim 22, characterized in that the constituted mask is frozen.

37. Process for the production of a product for pharmaceutical and/or cosmetic care according to claim 22, characterized in that the support of absorbent material is impregnated with a preparation including at least one active principle and is packaged in sealed packaging.

38. Process for the production of a product for pharmaceutical and/or cosmetic-care according to claim 37, characterized in that the product is frozen before packaging.
